(19) United States
(12) Patent Application Publication
(10) Pub. No.: US 2008/0292719 A1
(43) Pub. Date: Nov. 27, 2008

(54) Method of Treating Metabolic Syndrome

(76) Inventors: Igor Anatolievich Pomytkin, Moscow (RU); Sergey Pavlovich Soloviev, Moscow (RU)

Correspondence Address: NOTARO AND MICHALOS, 100 DUTCH HILL ROAD, SUITE 110, ORANGEBURG, NY, 10962-2100, US

(21) Appl. No.: 12/096,510

(22) PCT Filed: Dec. 12, 2005
(86) PCT No.: PCT/RU2005/000641

§ 371 Date: Aug. 13, 2008

(51) Int. Cl. A61K 33/00; C01B 5/00; A61P 3/10

(52) U.S. Cl. 424/600; 423/580.1

(57) ABSTRACT

The invention relates to the method of treating metabolic syndrome in a mammal in need thereof comprising a step of administering to said mammal an effective amount of water comprising from 99.76 to 99.99 molecular % of isotopologue ¹H₂¹⁶O. Further, invention relates to the medical food for treating metabolic syndrome in a mammal in need thereof which comprises water comprising from 99.76 to about 99.99 molecular % of isotopologue ¹H₂¹⁶O. Further, invention relates to the use of water comprising from about 99.76 to about 99.99 molecular % of isotopologue ¹H₂¹⁶O for the manufacture of a medical food for treating metabolic syndrome in a mammal in need thereof.

### TECHNICAL FIELD

The present invention is in the field of healthcare. More specifically, this invention relates to the method for treating metabolic syndrome.

### BACKGROUND ART

Metabolic syndrome, also called insulin resistance syndrome or syndrome X, is a cluster of risk factors responsible for much of the excess cardiovascular disease morbidity, wherein insulin resistance plays the role of the underlying pathophysiological defect. Its clinical identification is based on measures of abdominal obesity, atherogenic dyslipidemia, raised blood pressure, and glucose intolerance. The metabolic syndrome is a precursor to type 2 diabetes and a strong risk factor for coronary heart disease (CHD) and stroke. Schulze M B and Frank B H. Diabetes Care 27:613-614, 2004.

A report from the National Cholesterol Education Program-Adult Treatment Panel (NCEP-ATP III) identified metabolic syndrome as an independent risk factor for cardiovascular disease and stroke and considered it an indication for intensive lifestyle modification. The NCEP-ATP III has created an operational definition of metabolic syndrome: the co-occurrence of any three of a set of five metabolic abnormalities such as hypeitriglyceridemia, abdominal obesity, high blood pressure, low HDL cholesterol, and high fasting glucose. National Institutes of Health: Third Report of the National Cholesterol Education Program Expert Panel on Detection, Evaluation, and Treatment of High Blood Cholesterol in Adults (Adult Treatment Panel III). Executive Summary. Bethesda, Md.: National Institutes of Health, National Heart Lung and Blood Institute, 2001 (NIH publication no. 01-3670).

Metabolic syndrome, defined by an expert panel of the World Health Organization in 1998, includes insulin resistance, abdominal obesity, elevated blood pressure, and lipid abnormalities (i.e., elevated levels of triglycerides and low levels of high-density lipoprotein [HDL] cholesterol). Diagnostic criteria for metabolic syndrome according to the WHO include insulin resistance plus two of the following components: abdominal/central obesity, hypertriglyceridemia, low HDL cholesterol, high blood pressure, high fasting glucose, and microalbuminuria. Alberti K G, Zimmet P Z. Definition, diagnosis and classification of diabetes mellitus and its complications. Part 1: diagnosis and classification of diabetes mellitus, provisional report of a WHO consultation. Diabet Med 1998; 15:539-53.

Based on 2000 data and ATP III definition, 47 million US residents have the metabolic syndrome. Ford E S et al. Prevalence of the Metabolic Syndrome Among US Adults: Findings From the Third National Health and Nutrition Examination Survey. JAMA. 2002; 287:356-359.

Effective prevention and treatment metabolic syndrome involves a multifaceted approach focused on the individual components of the syndrome as well as modifications of lifestyle and diet.

Insulin sensitizers (thiazolidinediones and metformin) and weight loss medications are classes of drugs for treating metabolic syndrome. These medications have serious side effects and complications. Thus, there is a great need for a safe, effective agent for treating metabolic syndrome with little or no complications and side effects.

It is known that natural water is a composition of nine water isotopologues (¹H₂¹⁶O, ¹H₂¹⁷O, ¹H₂¹⁸O, ¹H²H¹⁶O, ¹H²H¹⁷O, ¹H²H¹⁸O, ²H₂¹⁶O, ²H₂¹⁷O, ²H₂¹⁸O) formed by stable isotopes of hydrogen (¹H and ²H) and oxygen (¹⁶O, ¹⁷O, ¹⁸O), wherein the level of light water isotopologue ¹H₂¹⁶O is about 99.7317 molecular % (Vienna Standard Mean Ocean Water, VSMOW), and wherein total level of all eight heavy isotopologues comprising at least one heavy isotopes ²H, ¹⁷O, or ¹⁸O is about 0.2683% (e.g. 0.199983% ¹H₂¹⁸O, 0.0372% ¹H₂¹⁷O, 0.031069% ¹H²H¹⁶O, 0.0000623% ¹H²H¹⁸O, and 0.0000116% ¹H²H¹⁷O). Rothman et al., J. Quant. Spectrosc. Radiat. Transfer, 1998, 60, 665. Rothman et al., J. Quant. Spectrosc. Radiat. Transfer, 2003, 82, p. 9. The abundance of water isotopologues in natural water slightly varies on Earth district and climatic conditions and is expressed typically as the deviation, δ, relative to the international VSMOW standard. The Earth water maximally enriched by major light water isotopologue ¹H₂¹⁶O was founded in Antarctica (Standard Light Antarctic Precipitation, SLAP), wherein said δ-values of residual heavy isotopes are δ²H −415.5‰, δ¹⁷O −28.1‰, and δ¹⁸O −53.9‰ that corresponds to the 99.757% level of light water isotopologue ¹H₂¹⁶O. R. van Trigt, Laser Spectrometry for Stable Isotope Analysis of Water Biomedical and Paleoclimatological Applications, 2002, Groningen: University Library Groningen, p. 50. Thus, water with the abundance of light water isotopologue 1H₂¹⁶O more than 99.757 molecular % is not found in nature.

Deuterium depleted water (DDW) is known from the art and is prepared from natural water by industrial procedures providing depletion of heavy isotopologues comprising deuterium, predominantly of ¹H²H¹⁶O (HOD). Since total levels of deuterium-comprising isotopologues in water is below 0.031 molecular %, complete depletion of natural water of deuterium-comprising isotopologues provides water enriched by light water isotopologue ¹H₂¹⁶O to the level never more than 99.76 molecular %. Thus, water with level of light water isotopologue ¹H₂¹⁶O more than 99.76% is unknown from the art and can be prepared in industrial scale by methods providing depletion of natural water of heavy isotopologues comprising isotopes ¹⁷ O and ¹⁸O.

We discovered that water with isotopologue ¹H₂¹⁶O level more than 99.76 molecular % is useful for treating metabolic syndrome in mammals in need thereof.

It is an object of the present invention to provide a method of treating metabolic syndrome in a mammal in need thereof comprising a step of administering to said mammal an effective amount of water comprising from 99.76 to 99.99 molecular % of isotopologue ¹H₂¹⁶O.

It is an object of the present invention to provide a medical food for treating metabolic syndrome in a mammal in need thereof which comprises water comprising from about 99.76 to about 99.99 molecular % of isotopologue ¹H₂¹⁶O.

It is an object of the present invention to provide the use of water comprising from about 99.76 to about 99.99 molecular % of isotopologue ¹H₂¹⁶O for the manufacture of a medical food for treating metabolic syndrome in a mammal in need thereof.

### BRIEF DESCRIPTION OF THE DRAWINGS

FIG. 1 is a schematic side view of an apparatus for the manufacturing the water comprising from 99.76 to 99.99 molecular % of isotopologue ¹H₂¹⁶O.

### DISCLOSURE OF INVENTION

The present invention provides a method of treating metabolic syndrome in a mammal in need thereof comprising a step of administering to said mammal an effective amount of water comprising from 99.76 to 99.99 molecular % of isotopologue ¹H₂¹⁶O.

As used herein, the term “isotopologue” is in accordance with IUPAC Compendium of Chemical Terminology 2nd Edition (1997) and refers to a molecular entity that differs only in isotopic composition (number of isotopic substitutions), e.g. ¹H₂¹⁶O, ¹H₂¹⁶O, ¹H₂¹⁸O.

The water of the invention comprising from 99.76 to 99.99 molecular % of isotopologue ¹H₂¹⁶O can be prepared by a variety of industrial procedures. Such procedures include, but are not limited to, burning molecular hydrogen with molecular oxygen with desired low heavy isotope content, or industrial procedures providing purification of natural water of heavy isotopologues comprising heavy isotopes ²H, ¹⁷O, and ¹⁸O. Preferably, the water of the invention is prepared by highly-effective distillation of natural water.

According to present invention, the water of the invention comprises from 99.76 to 99.99 molecular % of isotopologue ¹H₂¹⁶O and up to 100 molecular % of residual isotopologues. As used herein, the term “residual isotopologues” refers to ¹H₂¹⁷O, ¹H₂¹⁸O, ¹H₂H¹⁶O, ¹H²H¹⁷O, ¹H²H¹⁸O, ²H₂¹⁶O, ²H₂¹⁷O, and ²H₂¹⁸O. In the invention, relative amounts of particular heavy isotopologues could vary depending upon the procedure of the preparing the water of the invention, but the total sum of residual isotopologues formed by heavy isotopes ²H, ¹⁷O, [symbol] ¹⁸O should not exceed 0.01 to 0.24 molecular %. The amounts of heavy isotopes in the residual isotopologues could vary from 0.01 ppm to 155 ppm for ²H, 1 to 360 ppm for ¹⁷O, and 1 to 2000 ppm for ¹⁸O, but the total sum of the residual isotopologues formed by these amounts of heavy isotopes should not exceed 0.01 to 0.24%.

In practicing the method of the invention, the effective amount of water comprising from 99.76 to 99.99 molecular % of isotopologue ¹H₂¹⁶O can be administered in a variety of routes including oral (e.g. through gastrointestinal tract or oral mucosa), intranasal, topical, rectal, by inhalation spray, or parenteral (e.g. subcutaneous, intravenous, or intramuscular injections). Preferably, the effective amount water comprising from 99.76 to 99.99 molecular % of isotopologue ¹H₂¹⁶O is administered orally.

Preferably, the effective amount of the water comprising from about 99.76 to about 99.99 molecular % of isotopologue ¹H₂¹⁶O is 0.1 to 50 g/kg body weight of a mammal per day.

Preferably, the effective amount water comprising from 99.76 to 99.99 molecular % of isotopologue ¹H₂¹⁶O is administered for 1 day or longer. More preferably, the effective amount water comprising from 99.76 to 99.99 molecular % of isotopologue ¹H₂¹⁶O is administered for 7 to 30 days.

As used herein, the term “mammal” refers to any mammal. Nonexclusive examples of such mammals include, but are not limited to, animals such as a dog, a cat, and a horse and a human. Preferably, the mammal is a human.

As used herein, the term “metabolic syndrome” refers to a cluster of risk factors responsible for much of the excess cardiovascular disease morbidity, wherein insulin resistance plays the role of the underlying pathophysiological defect. These risk factors include, but are not limited to, hypertriglyceridemia, abdominal obesity, high blood pressure, or low HDL cholesterol.

As used herein, the term “treating” means preventing a metabolic syndrome from occurring in a mammal that may be predisposed to the metabolic syndrome but has not yet been diagnosed as having it; inhibiting metabolic syndrome, e.g., arresting its development; relieving metabolic syndrome, e.g., causing regression of the condition of metabolic syndrome; slowing progression of metabolic syndrome; and/or attenuating metabolic syndrome.

In practicing the method of the invention, the effective amount of water comprising from 99.76 to 99.99 molecular % of isotopologue ¹H₂¹⁶O isotopologues can be administered in a variety of different dosage forms, i.e., they may be formulated in the form of solutions, spray, liquid aerosols, elixirs, syrups, and the like. Ingredients that can be used for preparing dosage forms of the invention may include, but are not limited to, buffering agents (such as phosphate buffer, carbonate buffer, tris buffer, tartrate buffer, borate buffer, acetate buffer, succinate buffer, or maleate buffer), colorants, flavorants, preservatives, antioxidants, surfactants, and etc.

In practicing the method of the invention, the effective amount of water comprising from 99.76 to 99.99 molecular % of isotopologue ¹H₂¹⁶O can be administered stepwise or simultaneously with other agents for treating metabolic syndrome. Such agents include, but are not limited to, agents for treating hypertension, agents for lowering elevated HDL cholesterol, agents for lowering elevated triglycerides, and insulin sensitizers like metformin.

Further, the present invention provides a medical food for treating metabolic syndrome in a mammal in need thereof which comprises water comprising from about 99.76 to about 99.99 molecular % of isotopologue ¹H₂¹⁶O.

Further, the present invention provides the use of water comprising from about 99.76 to about 99.99 molecular % of isotopologue ¹H₂¹⁶O for the manufacture of a medical food for treating metabolic syndrome in a mammal in need thereof.

Preferably, the medical food for treating metabolic syndrome is drinking water or beverage. Preferably, the medical food of the invention is drinking water manufactured by saturation of the water of the invention with carbon dioxide or/and inorganic salts typically abandoned in natural drinking water. The examples of such salts include, but are not limited to, sodium chloride, sodium bicarbonate, calcium chloride, magnesium sulfate, etc.

Because of treating metabolic syndrome, the invention is particularly useful for preventing risk of cardiovascular diseases in mammals, preferably humans.

The following examples are presented to demonstrate the invention. The examples are illustrative only and are not intended to limit the scope of the invention in any way.

### EXAMPLE 1

This example demonstrates the method for producing the water of the invention.

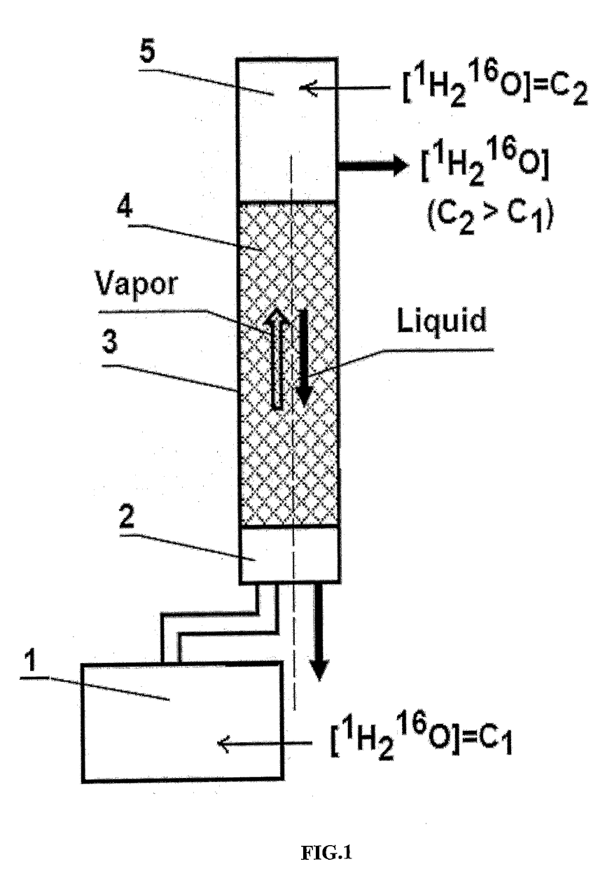

Water comprising from 99.76 to 99.99 molecular % of isotopologue ¹H₂¹⁶O is prepared by distillation of natural water comprising 99.73% of isotopologue ¹H₂¹⁶O with using the apparatus of FIG. 1 under temperature 60° C. and pressure 0.2 bars. The process of the distillation comprises evaporating natural water comprising 99.71% (C₁) of isotopologue ¹H₂¹⁶O in boiling means 1 to produce water vapor; supplying the water vapor to the bottom 2 of distillation column 3; carrying out vapor-liquid contact between a descending liquid and an ascending vapor mainly on the surface of the contact device 4 (e.g. structured or random packing) within the distillation column, at which time the liquid and the vapor flow in mutually opposite directions over the surface of the contact device along a main flow direction which is along a direction of the column axis; condensing water vapor with concentration of isotopologue ¹H₂¹⁶O from 99.76% to about 99.99% (C₂) on condenser 5 installed on upper bound of the distillation column 3; and collecting a part of condensate as condensed water comprising from 99.76% to 99.99% of isotopologue ¹H₂¹⁶O (C₂>C₁). Water comprising from 99.76 to 99.99 molecular % of isotopologue ¹H₂¹⁶O is used in the examples of the invention.

### EXAMPLE 2

This example demonstrates the method for treating metabolic syndrome.

Male Wistar rats received fructose-rich diet (66% fructose of total calorie intake) to induce insulin resistance and metabolic syndrome. The rats were assigned into two groups: a control rats (n=10) and experimental rats (n=10). Experimental rats received water of the invention (99.80% of isotopologue ¹H₂¹⁶O) as water for drinking. Control rats received control water with natural isotopes content (99.73 molecular % of isotopologue ¹H₂¹⁶O) as water for drinking. Both types of water were received ad libitum. Blood pressure, triglycerides, and fasting insulin (as an index of insulin resistance) were measured at 28 day of the experiment. Data are presented in Table 1. Table 1 demonstrates that water of the invention is effective for treating metabolic syndrome as compared to control.

### EXAMPLE 3

Table 2 demonstrates the medical food (drinking water) for treating metabolic syndrome.

The medical food as described in Table 2 was manufactured as follows: salts (calcium chloride, magnesium chloride, and sodium bicarbonate) in amounts shown in Table 2 were dissolved in the water (99.99 molecular % of isotopologue ¹H₂¹⁶O) under room temperature and, then, resulted product was bottled in bottles of 330 ml volume.

### EXAMPLE 4

This example demonstrates medical food for treating metabolic syndrome.

The medical food (beverage) is manufactured by saturation of the composition of example 3 with carbon dioxide and bottling the final product.

### EXAMPLE 5

This example demonstrates that drinking beverage of example 4 resulted in treating metabolic syndrome in overweight humans.

Two overweight male humans with mean BMI 29.0 kg/m², blood pressure mean 135/85 mm Hg, and triglycerides mean 1.9 mmol/l have received 100 ml/day of the beverage of example 4 at 60 min after eating for a period of 30 days. At the end of the experiment the mean BMI was reduced to the 26.8 kg/m², blood pressure mean was reduced to 125/80 mm Hg, and triglycerides mean was reduced to 1.5 mmol/l.

TABLE 1
Metabolic Parameters

Mean ± SD (n = 10)

| Parameter | Control | Experimental |
|---|---|---|
| Fasting plasma insulin, pmol/l | 310 ± 53 | 232 ± 22* |
| Blood pressure, mm Hg | 132 ± 6 | 117 ± 3* |
| Triglycerides, mmol/l | 2.3 ± 0.5 | 1.6 ± 0.1* |

*Denotes statistically significant difference of control (p < 0.05)

TABLE 2
Medical food for treating metabolic syndrome.

| | Content, |
|---|---|
| | weight % |
| Water (99.99 molecular % of isotopologue ¹H₂¹⁶O) | 99.953 |
| Calcium chloride | 0.015 |
| Magnesium chloride | 0.007 |
| Sodium bicarbonate | 0.025 |

We claim:

1. A method of treating metabolic syndrome in a mammal in need thereof comprising a step of administering to said mammal an effective amount of water comprising from 99.76 to 99.99 molecular % of isotopologue ¹H₂¹⁶O.

2. The method according to claim 1, wherein the effective amount water comprising from 99.76 to 99.99 molecular % of isotopologue ¹H₂¹⁶O is administered orally.

3. The method according to claim 1, wherein the effective amount of the water comprising from about 99.76 to about 99.99 molecular % of isotopologue ¹H₂¹⁶O is 0.1 to 50 g/kg body weight of a mammal per day.

4. The method according to claim 1, wherein the effective amount water comprising from 99.76 to 99.99 molecular % of isotopologue ¹H₂¹⁶O is administered for 1 day or longer.

5. The method according to claim 1, wherein the mammal is a human.

6. A medical food for treating metabolic syndrome in a mammal in need thereof which comprises water comprising from about 99.76 to about 99.99 molecular % of isotopologue ¹H₂¹⁶O.

7. The medical food according to claim 6, wherein the mammal is a human.

8. The use of water comprising from about 99.76 to about 99.99 molecular % of isotopologue ¹H₂¹⁶O for the manufacture of a medical food for treating metabolic syndrome in a mammal in need thereof.

9. The use according to claim 8, wherein the mammal is a human.
